(19) United States
(12) Patent Application Publication
(10) Pub. No.: US 2011/0313295 A1
(43) Pub. Date: Dec. 22, 2011

(54) Fluorescence Method and System

(76) Inventors: Desmond Smith, Livingston (GB); Dmitry Gakamsky, Livingston (GB)

(21) Appl. No.: 13/147,930

(22) PCT Filed: Feb. 25, 2010
(86) PCT No.: PCT/GB2010/000332

§ 371 Date: Sep. 1, 2011

(30) Foreign Application Priority Data
Feb. 26, 2009 (GB) 0903274.9

(51) Int. Cl. A61B 6/00

(52) U.S. Cl. 600/476

(57) ABSTRACT

A method for detecting changes in a human or animal eye is provided. The method includes illuminating the eye or at least a part thereof, in particular the lens and/or cornea, using light at a red edge excitation wavelength; detecting fluorescence caused by the red edge excitation and using the detected fluorescence to detect or identify structural defects or changes in the eye.

### FIELD OF THE INVENTION

The present invention relates to a non-invasive method and system for detecting changes indicative of cataract or cataract formation, or other medical conditions, using fluorescence of a fluorophore, for example tryptophan, in the ocular lens. The method is applicable to in vivo detection of early changes in the eye lens structure. It can be also used for in vitro drug screening.

### BACKGROUND OF THE INVENTION

The ocular lens grows continuously throughout life. The major components of the lens are highly stable and water soluble crystallin proteins. Significant molecular changes accumulate in the protein over its very long life span, which are caused by chemical, photochemical and environmental factors. The changes lead to protein misfolding, denaturation and aggregation and hence structural defects. The defects in the lens structure can be caused by aging or concomitant diseases, e.g. diabetes, see Truscott, R. J. W. Protein misfolding, aggregation, and conformational diseases, Uverski V N and Fink A (eds.), pp. 435-447 (Springer, 2007). Hence, a non invasive method for detecting and following very early changes in the lens structure could help in making a prognosis on surgical treatment of cataract and detection of changes indicative of concomitant diseases.

Currently cataract detection is based on measurements of lens opacity by light scattering. This can be done using a conventional slit lamp based technique or other more complex methods, such as Optical Coherence Tomography etc, see Konstantopoulos, A., Hossain, P. & Anderson, D. F. Recent advances in ophthalmic anterior segment imaging: a new era for ophthalmic diagnosis? Br. J. Ophthalmol. 91, 551-557 (2007). A limitation of these scattering techniques is that the sizes of the structural defects must be comparable with the wavelength of light used, i.e. ca 400-600 nm. Therefore, methods based on light scattering only allow detection of large-scale changes in the ocular lens.

U.S. Pat. No. 5,203,328 describes a method for noninvasively diagnosing diabetes mellitus and cataracts using a fluorescent response. Diagnoses are made by illuminating ocular lens tissue with a narrow-band light source at a selected wavelength, detecting the backscattered radiation intensity at the peak of a non-tryptophan fluorescence, and normalizing the detected value with the intensity of its Rayleigh component. This method uses co-called auto-fluorescence of the lens, i.e. emission of fluorescent products accumulated in the lens over time.

Various other fluorescence techniques have been proposed to investigate the eye, for example as described by Van den Berg, T. J. in the article “Quantal and visual efficiency of fluorescence in the lens of the human eye”, Invest Ophthalmol. Vis. Sci. 34, 3566-3573 (1993) and by Bessems, G. J., et al in the article “Non-tryptophan fluorescence of crystallins from normal and cataractous human lenses” Invest Ophthalmol. Vis. Sci. 28, 1157-1163 (1987).

As well as non-tryptophan emission in the visible optical range of 400-550 nm, the eye lens exhibits UV emission in the 300-450 nm range caused by tryptophan fluorescence, which is associated with the proteins. Whilst tryptophan fluorescence is a promising technique, in vivo applications have been hindered by limited transparency of the cornea in the 280-310 nm spectral range where tryptophans can be excited most effectively and by the high concentration of crystallin proteins in the lens resulting in high optical density making it impossible to measure tryptophan emission from the interior of the lens. To illustrate this, consider that one crystalline molecule on average (averaged on alpha-, beta- and gamma-crystallins) contains four tryptophan residues, each of which has an extinction coefficient of 6,000 M⁻¹cm⁻¹, so that the extinction coefficient of one crystalline molecule is around 24,000 M⁻¹cm⁻¹. Total concentration of crystalline proteins in the lens is around 10 mM. Thus, the extinction coefficient of the lens is 24,000×0.001=240 M⁻¹cm⁻¹. This means that less than 0.5% of the excitation at a wavelength of 285 nm penetrates deeper than 0.1 mm.

### SUMMARY OF THE INVENTION

According to the present invention, there is provided a method for detecting changes in a human or animal eye comprising illuminating the eye to cause fluorophore, such as tryptophan, fluorescence; detecting features in the fluorescence associated with inhomogeneous broadening and using the detected features associated with inhomogeneous broadening to detect structural defects in the eye. The inhomogeneous broadening caused by changes or defects in the eye makes “red-edge” excitation spectrally available for various penetrations.

The method may involve using red edge excitation of the eye proteins to detect features due to inhomogeneous broadening. An advantage of this is that fluorescence from tryptophan side chains located in sites with different polarities in the lens can be detected.

The structural defects may be associated with protein damage, such as protein misfolding, denaturation and aggregation.

The method may involve predicting one or more conditions based on the detected features in the tryptophan fluorescence associated with inhomogeneous broadening. For example, the method may involve detecting features that are associated with cataracts or the formation or cataracts.

Tryptophan and non-tryptophan fluorescence anisotropy detected from different spatial points of the ocular lens allows determination of the size of protein complexes and hence can be used for characterisation of the lens structure.

According to another aspect of the invention, there is provided a system for detecting changes in a human or non human eye comprising means for illuminating the eye using red edge excitation to cause fluorescence of a fluorphore, for example tryptophan; a detector for detecting features in the tryptophan fluorescence associated with inhomogeneous broadening and means for using the detected features associated with inhomogeneous broadening to detect structural defects in the eye.

Detecting features due to inhomogeneous broadening involves detecting a shift in the fluorescence and fluorescence anisotropy at different points.

The structural defects may be associated with protein damage, such as protein misfolding, denaturation and aggregation.

The system may be adapted to simultaneously measure tryptophan fluorescence and non-tryptophan fluorescence (auto-fluorescence).

The system may be adapted to predict one or more conditions based on the detected features in the tryptophan fluorescence associated with inhomogeneous broadening, for example cataracts.

The system may be adapted to illuminate the eye in vivo.

The system may include a tuneable interference filter.

According to another aspect of the invention there is provided a method for detecting structural defects or changes in a concentrated protein sample, such as crystallised or partially crystallised protein, comprising illuminating the protein sample using red edge excitation of a fluorphore, for example tryptophan; detecting features in the fluorescence associated with inhomogeneous broadening and using the detected features associated with inhomogeneous broadening to detect structural defects or changes.

According to a further aspect of the invention, there is provided a method for detecting structural defects or changes in a concentrated protein sample, such as crystallised or partially crystallised protein, the method involving illuminating the sample using light at a red edge excitation wavelength; detecting fluorescence caused by the red edge excitation and using the detected fluorescence to detect or identify structural defects or changes. Defects or changes may be detected by the presence of a red edge shift in the emission spectrum.

According to yet another aspect of the invention there is provided a screening method for identification of biologically active compounds based on monitoring changes in fluorescence, for example tryptophan fluorescence, in the ocular lenses or in protein samples caused by application of screening compounds that uses a method or system according to any of the above mentioned aspects of the invention.

### BRIEF DESCRIPTION OF DRAWINGS

Various aspects of the invention will be described now by way of example only and with reference to the accompanying drawings, of which:

FIG. 1 shows a normalised excitation (black) and emission spectra (blue, 285 nm excitation, red, 315 nm excitation) of 1 mg/ml α-crystallin/PBS solution at 25° C. recorded in a FLS920 spectrometer (Edinburgh Instruments);

FIG. 2 shows fluorescence spectra of a folded (blue) and partially denatured (unfolded) (red) β-crystallin at 285 nm;

FIG. 3 shows positions of the shift in the maximum of the emission spectra of 0.75 mg/ml β-crystallin tryptophan emission as a function of three different sample conditions with shifts of ˜18 nm demonstrated when the conditions change from an aqueous solution to the application of urea which denatures the beta-crystallins;

FIG. 4 is a schematic layout of a dual axis confocal experimental setup for tryptophan fluorescence and fluorescence anisotropy measurements in the ocular lens;

FIG. 5 (a) shows corrected fluorescence spectra of a normal and irradiated pig lens at 305 nm excitation;

FIG. 5 (b) shows corrected fluorescence spectra of a normal and UV irradiated pig lens at 315 nm excitation;

FIG. 6 is a plot of 440 nm-band/tryptophan intensities as a function of excitation wavelength for a UV-irradiated lens (red), normal lens (blue), and the ratio of the two (black), and

FIG. 7 shows fluorescence spectra (not spectrally corrected) and fluorescence anisotropy spectra of the normal (blue curves) and UV irradiated lenses (red curves) measured at 305 nm excitation.

### DETAILED DESCRIPTION OF DRAWINGS

The present invention provides a system and method for detecting conditions relating to eye defects in the lens or cornea, such as cataracts or the possible formation of cataracts, using the so-called red-edge excitation shift. This is a shift in the wavelength of emission caused by a shift in the excitation wavelength towards the long wavelength or red edge of the absorption band. This is explained on the basis of inhomogeneous broadening of electronic spectra.

The red edge excitation technique is known and is been described in, for example, Demchenko, A. P. “Red-edge-excitation fluorescence spectroscopy of single-tryptophan proteins. Eur. Biophys J. 16, 121-129 (1988)”. Examples of inhomogeneous broadening of electronic spectra of dye molecules are described by Nemkovich N A, et al in “Topics in Fluorescence Spectroscopy” Lakowicz, J. R. (ed.), pp. 367-409 (Plenum, New York, 1991) and Gakamsky, D. M. et al. in “Selective laser spectroscopy of 1-phenylnaphthylamine in phospholipid membranes” Biophys. Chem. 42, 49-61 (1992).

The red-edge excitation method for studying protein conformation is based on the dependence of tryptophan fluorescence spectra on excitation wavelength. This dependence is caused by inhomogeneous broadening of tryptophan electronic spectra, which means that the 0-0 electron transition frequency varies due to fluctuations in microenvironment caused by molecular dynamics of a protein. Also different residues of a multi-tryptophan protein are situated in environments with different polarity, which depends on the protein sequence and three-dimensional structure. As a result, total emission and absorption spectra of a protein are convolutions of homogeneous absorption or emission spectra of tryptophan with a distribution function of the 0-0 transition frequency. Both the florescence of tryptophans and non-tryptophan fluorescence (auto-fluorescence) can be used to detect structural changes in proteins. At 320 nm excitation, fluorescence of tryptophan and also auto-fluorescence can be excited.

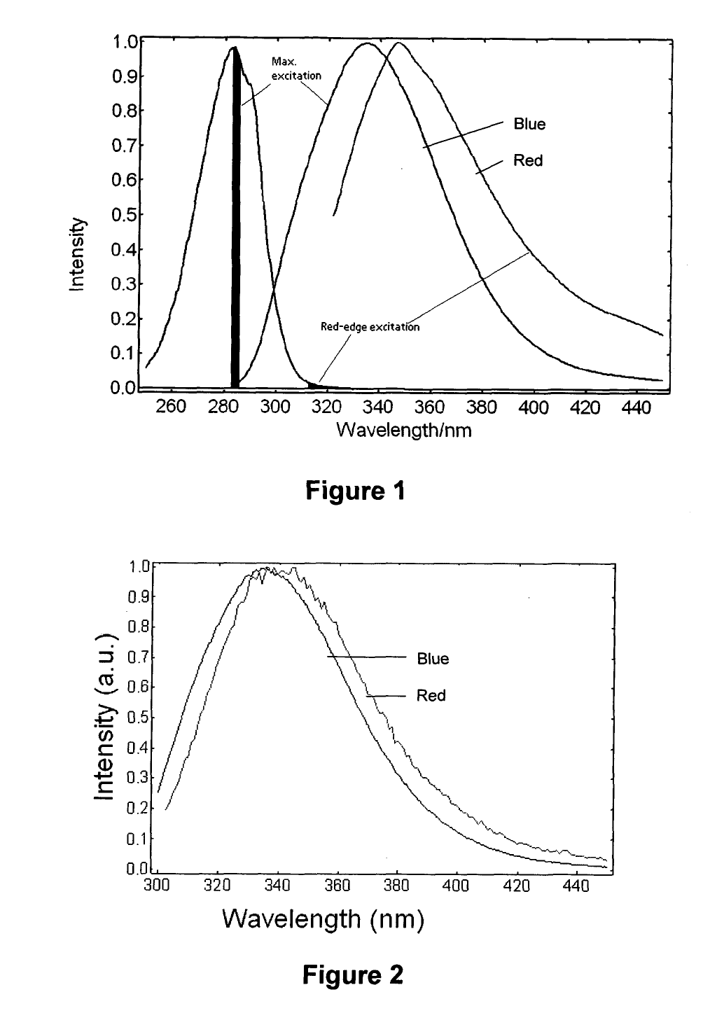

An important feature of tryptophan homogeneous absorption spectra is a very steep red slope: the extinction coefficient at 285 nm drops down to 5% of the maximal value at 295 nm and less than 1% at 310 nm. The steepness of the red slope allows spectrally selective excitation of the red-shifted fraction of tryptophans, situated in the most polar environment, which are also situated in spatially different areas of a protein. FIG. 1 illustrates red-edge excitation with α-crystallin/PBS solution. The major fraction of tryptophan residues is excited at maximum (blue line). A “red-shifted” fraction of tryptophans is selectively excited at 315 nm (red line).

The eye lens contains a very high concentration of crystallin molecules (−10 mM) comprising on average four tryptophan residues. Hence, the optical density of a 1 mm layer of the lens at 285 nm excitation is such that more than 99% of excitation at 285 nm wavelength will be absorbed by a 0.1 mm surface layer of the lens. In contrast, crystallin extinction at 315-320 nm excitation comprises ca ˜0.5% of that at the maximum and therefore the optical density of the lens will be such that only ˜3% of the excitation will be absorbed by 0.1 mm layer and ˜25% by 1 mm layer of the lens. Therefore, red-edge excitation allows detection of tryptophan fluorescence from different spatial points in the lens.

Three goals can be achieved by using red-edge excitation. First, this spectral range allows the excitation light to reach the lens through the cornea, which is transparent at wavelengths longer than 320 nm (see Ambach, W. et al. Spectral transmission of the optical media of the human eye with respect to keratitis and cataract formation. Doc. Ophthalmol. 88, 165-173 (1994) and van den Berg, T. J. & Tan, K. E. Light transmittance of the human cornea from 320 to 700 nm for different ages. Vision Res. 34, 1453-1456 (1994)). Second, it allows excitation of the lens interior and so allows access to structural information through the lens. Third, it allows excitation of the most red-shifted fraction of tryptophan residues located in highly polar microenvironment, which is a function of protein conformation.

Fluorescence spectra of folded (blue) and partially denatured (unfolded) (red) β-crystallin at 285 nm excitation are shown in FIG. 2. This shows that the number of tryptophans situated in a polar environment is increasing in the denatured protein and as a result the spectrum exhibits a red shift. Red-edge excitation at 315 nm shows fluorescence spectral shifts due to denaturing of the protein by addition of 8M of urea in the solution containing protein from bovine eye lenses. This illustrates sensitivity of the method to conformation of the ocular lens protein.

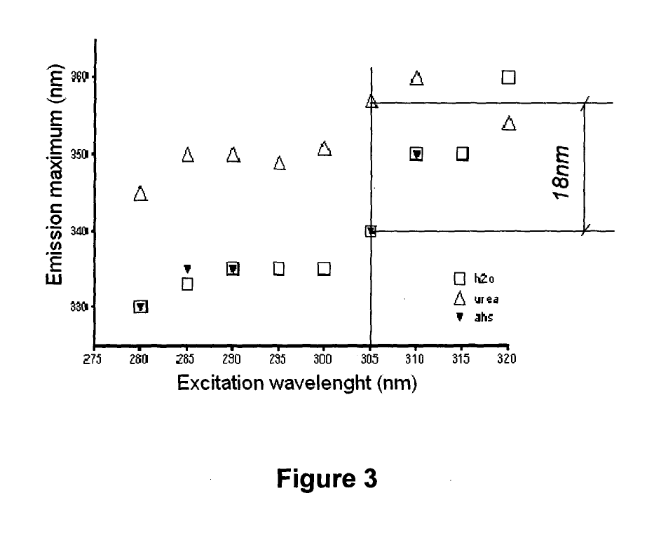

The peak positions of the emission spectra are shown in FIG. 3, where the symbols are: □ normal conditions, Δ denaturing conditions (PBS/8M urea), ▾ aqueous humor substitute. Under protein denaturing conditions, with more tryptophan residues exposed to the aqueous environment, there is a red shift of emission maximum of 18 nm in red edge excitation seen at 305 nm. This shows that the concentration of the polar fraction depends upon the protein conformation.

Due to the possibility of penetrating into the interior of the lens, red-edge excitation allows fluorescence anisotropy measurements throughout the lens. Fluorescence anisotropy measurements involve exciting a fluorophore with polarised radiation and monitoring the polarisation of the resultant emitted light. Tryptophan fluorescence anisotropy depends on molecular mass of a protein and hence can be used for measuring protein aggregation, as described by Gakamsky, D. M., et at in “Selective steady-state and time-resolved fluorescence spectroscopy of an HLA-A2-peptide complex”. Immunol. Lett. 44, 195-201 (1995). This is thought to be a cause of cataract formation. Thus, anisotropy can be used as a parameter characterising the lens structure.

Using red-edge excitation, for example at an excitation wavelength of 320 nm, non-tryptophan fluorescence of the lens is also excited. The latter is red-shifted from emission of tryptophans with maximum at ca. 440 nm. As the non-tryptophan fluorescence also correlates with the lens structure, its simultaneous measurement with the tryptophan emission allows a ratio-metric characterisation of the lens.

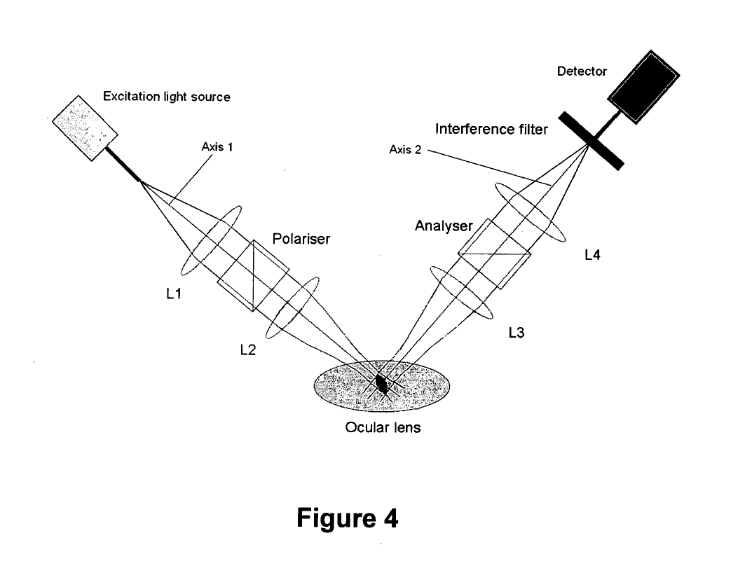

FIG. 4 shows an example of a dual axis confocal experimental setup for tryptophan fluorescence and fluorescence anisotropy measurements in the ocular lens. This has an excitation source that emits light in the range 315 nm-325 nm. As noted before, red-edge excitation wavelengths, for example 315 nm-325 nm, can penetrate into the lens interior and excite tryptophan fluorescence. The excitation source can work in a pulse mode and provide intensity and lifetime measurements. Light from the source passes along an emission path on which are positioned a first convex lens that collimates the light; a polariser that polarises the light and a second convex lens that directs the light onto the ocular lens that is under investigation.

Fluorescence from specific areas of interest of the ocular lens is emitted along an emission path on which are positioned a convex lens; an analyser for analysing the polarisation of the emitted light; and a second emission path convex lens that directs the light through a tunable or changeable interference filter towards a detector. Fluorescence can be detected by moving the confocal detection volume across the lens. The detector can be fibre-coupled to supply additional spatial selection. The tuneable (wedge) or changeable interference filters are provided in the emission path to allow maximal light collection in measurements of tryptophan and non-tryptophan fluorescence and also Raman scattering. Intensities of non-tryptophan fluorescence and/or that of the Raman signal can be used as a reference to make quantitative measurements of tryptophan fluorescence. The polarizer in the excitation path and the analyzer in the emission path can rotate by 90 degrees to allow measurements of the parallel and perpendicular fluorescence components to calculate fluorescence anisotropy.

In use, excitation light is focused in a specific region of interest within the lens. This causes excitation of fluorescence of tryptophan residues and non-tryptophan emission in the selected region. The total fluorescence intensity and fluorescence intensity of the parallel and perpendicular fluorescence components of tryptophan in the range 350-450 nm are measured, and auto-fluorescence in the range 450-550 nm, as is the fluorescence time-responses. The lens structure is then characterised using a combination of fluorescence parameters, such as fluorescence intensities, fluorescence anisotropy and fluorescence lifetimes of tryptophan and non-tryptophan (auto-fluorescence) emissions.

Red-edge excitation of tryptophan fluorescence allows detection of conformational changes in crystallin proteins. Tryptophan fluorescence anisotropy measurements allow detection of concentration-driven protein aggregation. These findings suggest that tryptophan fluorescence from the ocular lens could be used for non-invasive detection of medical conditions, such as cataracts. To demonstrate the effectiveness of this technique, studies were undertaken to compare fluorescence emission from a normal and cataract lenses to see to what extent changes in the lens structure can be detected. Extracted pig ocular lenses were used as a model.

In order to induce conformational changes, a pig lens was placed in a standard 1 cm fused silica cuvette in Phosphate Buffered Saline (PBS) and irradiated for 12 hours by 300-320 nm light in a single monochromator FLS920 spectrometer equipped with a 60W Xenon lamp. The intensity of the irradiation light was 7 mW/cm². The irradiation created a characteristic non-transparent spot of ˜2 mm diameter in the lens body close to the surface of the side subjected to the UV irradiation. Fluorescence from the UV damaged area in the irradiated lens was compared with that from a control (normal) lens, which was maintained at identical conditions in dark.

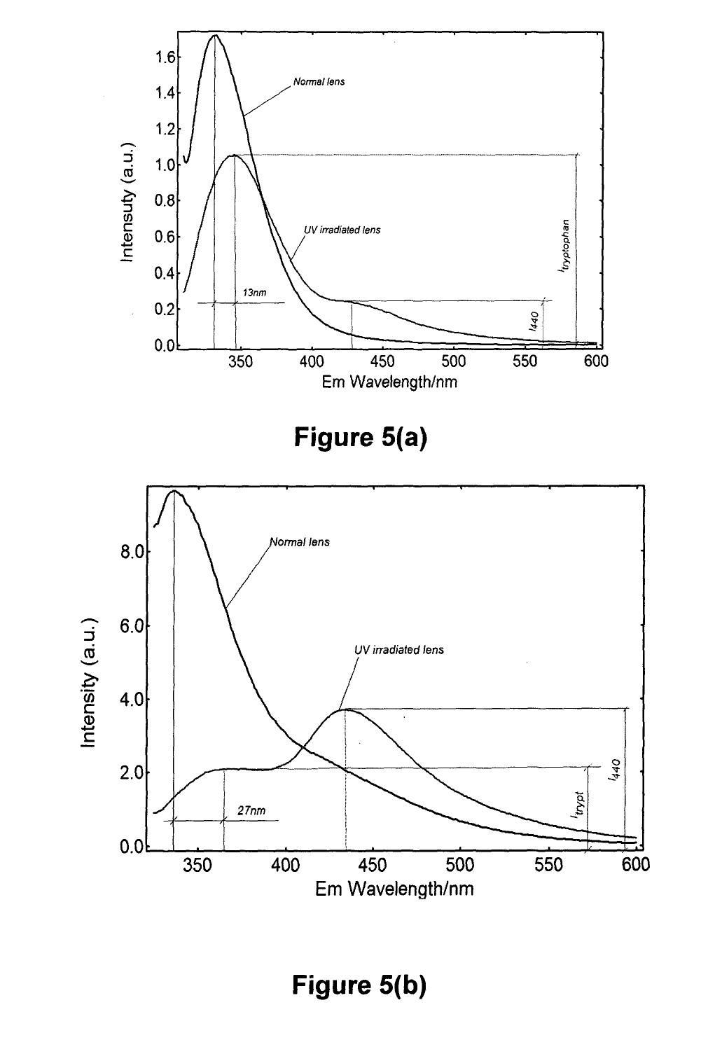

Fluorescence spectra of the irradiated and control ocular lenses were measured in a FLS920 spectrometer, Edinburgh Instruments, at 23° C. by using excitation in the 305 nm-325 nm range (spectral slits were 2 nm). Spectrally corrected fluorescence spectra at 305 nm and 315 nm excitations are shown in FIGS. 5(a) and (b). Emission spectrum of the normal lens at 305 nm excitation was mostly determined by tryptophan fluorescence, as can be seen from FIG. 5(a). A weak emission band with maximum at ca 440 nm was seen in the normal lens at 315 nm excitation, see FIG. 5(b). UV irradiation of the lens brought about an increase in the 440 nm intensity such that it was seen already at 305 nm excitation, see FIG. 5(a), and became dominant in emission of the irradiated lens at 315 nm excitation, see FIG. 5(b). The maximum of the fluorescence spectra of the irradiated lens was red-shifted by 13 nm at 305 nm excitation, see FIG. 5(a), and by 27 nm at 315 nm excitation from that of the non-irradiated lens, see FIG. 5(b). This result is consistent with earlier experiments with soluble crystallin samples at normal and denaturing (8M urea) conditions. Hence, the presence of a red shift in the emission spectra is indicative of structural defects or changes in the eye.

The presence of two bands in the ocular lens emission suggests a method for quantitative characterisation of the lens structure by the ratio of the peak intensities of the 440 nm and tryptophan bands:

$$F = \frac{{I}_{440}}{{I}_{tryptophan}} .$$

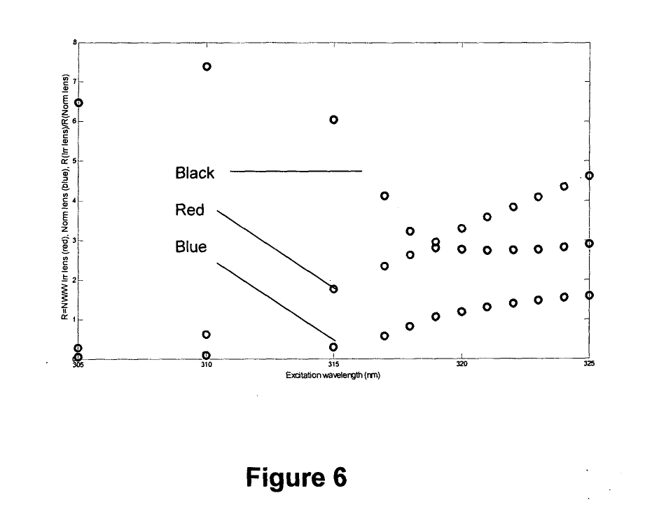

FIG. 6 shows F-parameters for the normal (blue circles) and UV irradiated lenses (red circles) as a function of excitation wavelength. The black curve is the ratio of F-parameters for the irradiated and normal lenses. It shows that the maximal change in F-parameter is achieved at 310 nm excitation. On the other hand, the maximal spectral shift of the tryptophan emission spectrum was observed at 315 nm. Hence, lens excitation at the red-edge of tryptophan absorption 310 nm-315 nm makes the above parameters the most sensitive and allows 3D mapping of the interior structure of the lens due to a sufficiently low extinction of the lens and cornea transparency.

Fluorescence anisotropy measurements of the irradiated and normal pig lenses were also carried out in the FLS920 spectrometer, Edinburgh Instruments, using a Glan-Thompson polariser and analyser. A fluorescence anisotropy spectrum is determined as follow:

$$r ( {λ}_{em} ) = \frac{{I}_{vv} ( {λ}_{em} ) - G \cdot {I}_{hv} ( {λ}_{em} )}{{I}_{vv} ( {λ}_{em} ) + 2 G \cdot {I}_{hv} ( {λ}_{em} )} ,$$

where Iᵥᵥ(λₑₘ) and Iᵥₕ(λₑₘ) are “parallel” and “perpendicular” fluorescence spectral components, and G is a correction factor accounting for differences in the intensity of the excitation light in the vertical (v) and horizontal (h) directions. The fluorescence anisotropy emission spectrum of tryptophan exhibits a slight declining tendency towards the red spectral range, which is caused by electron-vibration interaction.

Fluorescence anisotropy, r, allows determination of the rotational correlation time Φ of molecular tumbling of a fluorescent molecule:

$$r = \frac{{r}_{0}}{1 + \frac{τ}{φ}} ,$$

where r₀ is the limiting anisotropy value (ca 0.35 for tryptophan) and τ is fluorescence lifetime (decay of tryptophan fluorescence is not single exponential; the average lifetime can be taken as 5 ns). Rotational correlation time is a function of molecular mass and solvent viscosity, e.g. it is equal to 12 ns for a 10 kD protein in PBS. Intrinsic mobility or “wobbling” of the tryptophan side chain is another factor affecting tryptophan anisotropy in proteins. Amplitudes and rates of “wobbling” depend on location of tryptophans in the protein body. Usually “wobbling” rate constants are in the pico-second time-domain. Time-resolved fluorescence anisotropy measurements can resolve between the “tumbling” and “wobbling” depolarisation mechanisms, and molecular mass of a protein can be determined from the value of time-constant of the long anisotropy decay component. This parameter is determined by the size of crystallin complexes creating the lens structure. Average amplitude of “wobbling” and its characteristic time can be determined from the fast anisotropy decay component pre-exponential coefficient and time-constant respectively.

At non-denaturing conditions alteration in steady-state anisotropy values usually indicates changes in the rate of protein tumbling, i.e. changes in its molecular mass. In other words, decrease in steady-state anisotropy values usually suggests protein disaggregation.

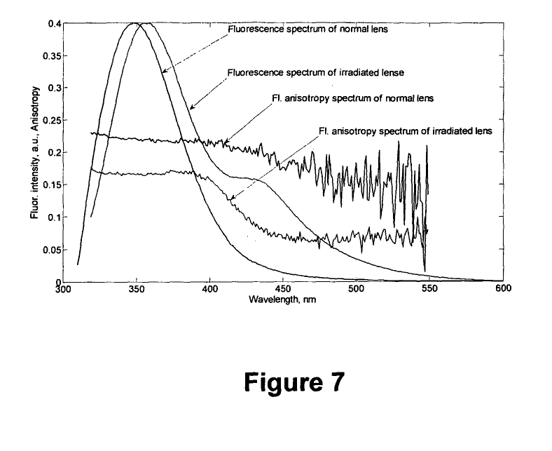

Fluorescence anisotropy and steady-state intensity spectra at 305 nm excitation of normal and irradiated lenses are shown in FIG. 7. In both cases, the anisotropy spectra exhibit a slight decline towards the red spectral range. Bends in the anisotropy spectra in the 400-430 nm range indicate the presence of two emission species, i.e. emissions of tryptophan with maximum at 340-360 nm and the non-tryptophan emission with maximum at ˜440 nm. The figure shows that anisotropy of tryptophan emission decreased by ˜5% and that of the 440 nm-band by ˜10%. These changes suggest that due to UV irradiation the lens structure becomes less “rigid” or the number of crystalline subunits in the protein aggregates decreases. The decrease in the tryptophan fluorescence anisotropy could also be caused in part by increase in the wobbling amplitude of tryptophan side chains of the UV irradiated proteins.

The results of the above studies show that tryptophan and non-tryptophan emission as well as fluorescence anisotropy can be used for detection of conformational changes in the ocular lens. Such changes are indicative of medical conditions, for example the formation of cataracts, and can be used for early stage, non-invasive detection.

A skilled person will appreciate that variations of the disclosed arrangements are possible without departing from the invention. For example, whilst the invention is described primarily with reference to detecting changes in the ocular lens, it could equally be used to detect changes in the cornea. To do this, the excitation wavelength would be less than for the lens, as the cornea is considerably thinner and thus absorbs at a shorter wavelength. Typically the excitation wavelength for detecting changes in the cornea would be in the range of 295-310 nm.

Although the invention is described in the context of detecting damage in the eye, typically in vivo, the ocular lens can be used as a platform for fluorescence-based screening. Extracted lenses can be used in search for drugs for treatment of cataract or other diseases caused by proteins misfolding, such as Alzheimer, Parkinson etc. The screening can be performed in a fluorimeter, a fluorescence plate/sample reader or similar fluorescence instrument. Equally, the method can be used to detect structural defects or changes in other environments where there are concentrated levels of protein. For example, the method could be used to detect defects or changes in crystallised or partially crystallised protein. Accordingly the above description of a specific embodiment is made by way of example only and not for the purposes of limitation. It will be clear to the skilled person that minor modifications may be made without significant changes to the operation described.

1. A method for detecting changes in a human or animal eye comprising illuminating the eye or at least a part thereof, in particular the lens and/or cornea, using light at a red edge excitation wavelength; detecting fluorescence caused by the red edge excitation and using the detected fluorescence to detect or identify structural defects or changes in the eye.

2. A method as claimed in claim 1 wherein detecting or identifying structural defects or changes involves identifying a red edge shift in the fluorescence.

3. A method as claimed in claim 1 wherein the excitation light is polarised and the method involves monitoring polarisation of the fluorescence.

4. A method as claimed in claim 3 wherein the detected fluorescence is fluoresence intensity and/or fluoresence anisotropy.

5. A method as claimed in claim 1 wherein the structural defects are associated with protein damage, such as protein misfolding, denaturation and aggregation.

6. A method as claimed in claim 3 wherein the fluorescence is tryptophan fluorescence.

7. A method as claimed in claim 6 comprising simultaneously measuring tryptophan fluorescence and non-tryptophan fluorescence.

8. A method as claimed in claim 2 wherein the red edge excitation light has a wavelength in the range 305 nm to 325 nm; in particular in the range 315 nm to 325 nm and more specifically in the range 310 nm to 315 nm.

9. A method as claimed in claim 8 wherein the excitation light has a wavelength in the range 295 nm to 310 nm.

10. A method as claimed in claim 1 comprising predicting one or more conditions based on the detected features in the fluorescence.

11. A method as claimed in claim 1 comprising illuminating the eye in vivo.

12. A screening method that uses a method according to claim 1.

13. A system for detecting changes in a human or non human eye comprising a light source for illuminating the eye or at least a part thereof, in particular the lens and/or cornea, with light at a red edge excitation wavelength; a detector for detecting fluorescence caused by the red edge excitation and means for using the detected fluorescence to detect or identify structural defects or changes in the eye.

14. A system as claimed in claim 13 wherein detecting or identifying structural defects or changes involves identifying a red edge shift in the fluorescence

15. A system as claimed in claim 13 wherein the excitation light is polarised and the detector is adapted to detect polarisation of the fluorescence.

16. A system as claimed in claim 13 wherein the structural defects are associated with protein damage.

17. A system as claimed in claim 13 wherein the fluorescence is tryptophan fluorescence.

18. A system as claimed in claim 17 adapted to simultaneously measure tryptophan fluorescence and non-tryptophan fluorescence (auto-fluorescence).

19. A system as claimed in claim 13 adapted to predict one or more conditions based on the detected features in the fluorescence.

20. A system as claimed in claim 14 wherein the red edge excitation light has a wavelength in the range 305 nm to 325 nm; in particular in the range 315 nm to 325 nm and more specifically in the range 310 nm to 315 nm.

21. A system as claimed in claim 20 wherein the excitation light has a wavelength in the range 295 nm to 310 nm.

22. A system as claimed in claim 13 adapted to illuminate the eye in vivo.

23. A system as claimed in claim 13 further comprising a tuneable interference filter.

24. A screening method for identification of biologically active compounds based on monitoring changes in fluorescence, for example tryptophan fluorescence, in the ocular lenses or in protein samples caused by application of screening compounds that uses a method according to claim 1.
